(19) United States
(12) Patent Application Publication
(10) Pub. No.: US 2012/0225852 A1
(43) Pub. Date: Sep. 6, 2012

(54) USE OF STEROID COMPOUNDS

(76) Inventors: Vasiliki Panoutsakopoulou, Athens (GR); Maria Angelakopoulou, Athens (GR); Theodora Calogeropoulou, Kifissia (GR); Achilleas Gravanis, Crete (GR); Lakovos Lazaridis, Athens (GR); Constantinos Neophytou, Nicosia (CY)

(21) Appl. No.: 13/395,381

(22) PCT Filed: Sep. 13, 2010
(86) PCT No.: PCT/GB2010/001727

§ 371 Date: May 16, 2012

(30) Foreign Application Priority Data
Sep. 11, 2009 (GB) 0916020.1
Jun. 3, 2010 (GB) 1009342.5

(51) Int. Cl. A61K 31/56; A61P 11/00; A61P 27/02; A61P 1/04; A61P 19/02; A61P 17/06; A61P 17/00; A61P 11/06; A61P 37/00

(52) U.S. Cl. 514/173; 514/178

(57) ABSTRACT

This invention pertains to the use of steroid compounds including spirosteroid analogues in treating, preventing or ameliorating the symptoms of inflammatory conditions. The steroid compounds are useful for treating a range of inflammatory conditions, including, but not limited to asthma, lung inflammation, retinal inflammatory conditions, autoimmune diseases such as rheumatoid arthritis, diabetes type I, systemic lupus erythematosus, ulcerative colitis and inflammatory bowel diseases and myopathies, as well as multiple sclerosis. The active compounds are represented by Formula I:

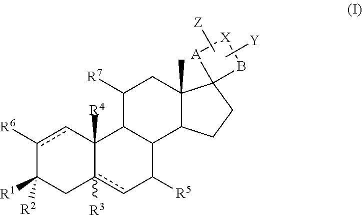

wherein R¹, R², R³, R⁴, R⁵, R⁶, R⁷, A, B, X, Y and Z are defined in the description of the invention.

### FIELD OF THE INVENTION

This invention pertains to the use of steroid compounds including spirosteroid analogues in treating, preventing or ameliorating the symptoms of inflammatory conditions, for example asthma. The mechanism of action on the immune system indicates that the steroid compounds are useful for treating a range of inflammatory conditions, including, but not limited to asthma, lung inflammation, retinal inflammatory conditions, autoimmune diseases such as rheumatoid arthritis, multiple sclerosis and systemic lupus erythematosus, inflammatory bowel diseases and myopathies.

### BACKGROUND OF THE INVENTION

Inflammation is an adaptive response triggered by a variety of noxious stimuli and conditions. Inflammation triggers the recruitment of leukocytes and plasma proteins to the affected tissue site. It underlies many of the human diseases associated with the immune system. The list of inflammatory conditions continues expanding to include common diseases initially not thought to be inflammatory, but degenerative. Inflammation is categorized as exogenous, causing the exogenous inflammation-associated diseases (infections, allergens, toxic exposure, drugs, chemicals, smoking, pollution, gluten, cholesterol, glucose) and endogenous (auto-inflammation vs autoimmune inflammation). Auto-inflammatory diseases (FMF, HIDS, TRAPS) are diseases of innate immunity, while autoimmune diseases (systemic lupus erythematosus, rheumatoid arthritis, multiple sclerosis, myasthenia gravis, aplastic anaemia, autoimmune haemolytic anaemia, refractory scleroderma, dermatitis, acquired pemphigus, Grave's disease, autoimmune hepatitis, psoriasis, Crohn's disease, ulcerative Colitis) are diseases of innate and adaptive immunity.

Immunity against pathogens is mediated through induction of antigen-specific T helper (Th) lymphocytes Th1, Th2 and Th17. Th1 immunity confers protection against intracellular pathogens and when excessive can lead to autoimmunity. Aberrant Th1- and Th17-cell activation can lead to autoimmunity, while excessive Th2-cell activation against environmental antigens may induce allergy and asthma. Th1 cells produce IFN-γ and activate MΦs to fight against intracellular pathogens. Th2 cells produce cytokines such as IL-4, IL-5 and IL-13 that stimulate mast cells and eosinophils and enhance IgE production by B cells. Th17 cells coordinate innate and adaptive immune responses against pathogens, such as fungi and bacteria. A different T cell subset, T regulatory cells (Treg), limits immune pathology by exuberant Th1, Th2 or Th17 cells. Activation and differentiation of Th immunity depends on interactions of Th cells with antigen presenting cells, such as dendritic cells (DCs), and cytokines play a critical role in this process.

Exuberant Th1, Th2 or Th17 immune responses are limited by mechanisms of suppression (immunosuppression). These mechanisms include the local secretion of cytokines such as TGF-β and direct cell contact through binding of cell surface molecules, such as CTLA-4 on suppressor T cells to CD80 and CD86 molecules on effector T cells. Suppression requires the appropriate colocalization of suppressor and effector T cells in different tissues and may involve the interference with T cell receptor signalling that triggers transcription factors important in regulating effector cell function. Treg cells display important suppressor activity. Treg cells play an indispensable role in maintaining immunological unresponsiveness to self-antigens and in suppressing excessive immune responses deleterious to the host. Tregs are produced in the thymus as a functionally mature subpopulation of T cells and can also be induced from naive T cells in the periphery. Recent research reveals the cellular and molecular basis of Treg development and function and implicates dysregulation of Tregs in immunological disease.

Glucocorticoids, which are endogenous steroids, are probably some of the most powerful anti-inflammatory drugs; however, these drugs can have many undesirable side effects (e.g., central obesity, hyperglycemia, osteoporosis) and their use must be tightly controlled.

Synthetic derivatives of dehydroepiandrosterone (DHEA), such as 5-androstene-16α-fluoro-17-one (HE2500) and certain natural metabolites have been shown to provide benefit in various animal models of autoimmune and metabolic diseases. But, like DHEA, the low potency and low oral bioavailability of these compounds suggested that they would have limited usefulness in humans. HE3286, a novel 17-ethynyl derivative of DHEA, exhibited up to 25% oral bioavailability in mice and in the DBA mouse model of collagen-induced arthritis (CIA); animals receiving oral treatment with HE3286 (50 mg/kg), beginning at onset of disease, showed significantly decreased CIA peak scores and daily severity of arthritis scores. HE3286 was not found to be immune suppressive in any of the classical models tested, including mitogen-induced proliferation, delayed-type hypersensitivity, or mixed lymphocyte reaction. Instead, benefit was associated with increases in numbers and function of CD4+CD25+FOXp3+CD127-regulatory T cells (T reg).

There is a sustained need for the development of new treatments for inflammatory conditions. Natural steroids such as DHEA possess important immunosuppressant properties in experimental animals. However, naturally occurring steroids are metabolised in humans into estrogens, androgens or progestins which exert generalized and important endocrine side effects, including hormone-dependent neoplasias (Front. Neuroendocrinol. 21, 1 (2000)), thus limiting their clinical use.

U.S. Pat. No. 3,365,475 (1968) discloses a process for the preparation of 17α-(3′-hydroxy-propyl)-4-androstene-3β,17β-diol which is useful in the preparation of steroidal 17-spirotetrahydrofurans which possess useful therapeutic properties as aldosterone inhibitors.

U.S. Pat. No. 4,026,918 (1977) describes the preparation of certain D-homosteroids that are said to have anti-inflammatory activity. (3β,11α,17α)-Spiro[androst-5-ene-17,2′-oxirane]-3,11-diol is disclosed as a chemical intermediate.

U.S. Pat. No. 4,054,563 (1977) discloses a process for the manufacture of certain 17-spiro-(2′-oxacyclopentane) steroid compounds that are said to be useful intermediates for preparing aldosterone antagonists.

WO 98/33506 discloses the use of certain compounds for inhibiting androgen synthesis, which are said to be useful in treating prostate cancer and benign prostatic hypertrophy. 17β,20β-Aziridinyl-pregn-5-en-3β-ol is one of the comparison compounds listed.

In the Journal of Medicinal Chemistry 10(4), 546-551 (1967), the steroidal cyclic ether of formula B below is mentioned as an intermediate in the preparation of steroidal compounds having antiestrogenic properties.

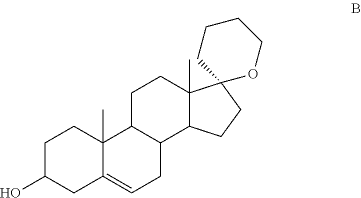

WO 2008/155534 discloses neurosteroid compounds and their use in treating neurodegenerative conditions relating to neuronal apoptosis or neuronal injury, including Alzheimer's disease and Parkinson's disease.

### SUMMARY OF THE INVENTION

In a first aspect, the present invention relates to a method of preventing or treating an inflammatory condition, comprising administering to a patient an effective amount of a compound of Formula I:

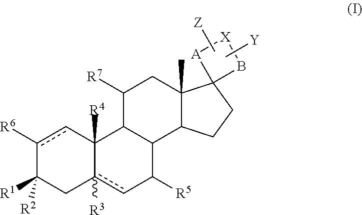

wherein R¹, R², R³, R⁴, R⁵, R⁶, R⁷, A, B, X, Y and Z are as defined in the detailed description below; or a pharmaceutically acceptable ester, salt or acid addition salt thereof.

Said inflammatory condition may, by way of example only, be any of asthma, lung inflammation, retinal inflammatory conditions, autoimmune diseases such as rheumatoid arthritis, diabetes type I, systemic lupus erythematosus, myasthenia gravis, aplastic anaemia, autoimmune haemolytic anaemia, refractory scleroderma, dermatitis, acquired pemphigus, Grave's disease, autoimmune hepatitis, psoriasis, Crohn's disease, ulcerative colitis and inflammatory bowel diseases and myopathies. Multiple sclerosis is another condition that may be treated in accordance with the invention.

In another aspect, this invention relates to a compound of Formula I, or a pharmaceutically acceptable ester, salt or acid addition salt thereof, for use in preventing or treating an inflammatory condition. Said condition may, for example, be any of those listed above.

In another aspect, this invention relates to the use of a compound of Formula I, or a pharmaceutically acceptable ester, salt or acid addition salt thereof, for the manufacture of a medicament for preventing or treating an inflammatory condition. Said condition may, for example, be any of those listed above.

In another aspect, this invention relates to the use of a compound of Formula I, or a pharmaceutically acceptable ester, salt or acid addition salt thereof, to control or suppress an immunological response of a human or non-human animal body.

In another aspect, this invention relates to the use of a compound of Formula I, or a pharmaceutically acceptable ester, salt or acid addition salt thereof, to control or suppress T-cell activity in a human or non-human animal body.

A better understanding of the invention will be obtained from the following detailed description of the article and the desired features, properties, characteristics, and the relation of the elements as well as the process steps, one with respect to each of the others, as set forth and exemplified in the description and illustrative embodiments.

### BRIEF DESCRIPTION OF THE DRAWINGS

FIG. 1 is a bar chart showing the effect of several steroid compounds on stimulated cultured mice lymph node cells, in an experimental study.

FIG. 2 comprises the results of an experimental study in the form of bar charts showing the effect of several steroid compounds on certain unprimed purified cultured mice CD4⁺ T cells (FIG. 2a), and their effect upon the proliferation and secretion of IL-2 and IFN-γ (FIGS. 2b and 2c respectively).

FIG. 3 is a bar chart showing the effect of several steroid compounds on stimulated cultured mice purified CD4⁺ T cells, in an experimental study.

FIG. 4 is a bar chart showing the effect of several steroid compounds on the numbers of CD4⁺ 7AAD⁻ cells in cultures after flow-cytometric analysis, in an experimental study.

FIG. 5 shows schematically a timeline for an experimental protocol for investigating the ability of several steroid compounds to protect against MOG peptide-induced experimental autoimmune encephalomyelitis (EAE).

FIG. 6 is a graph showing the effect over time of several steroid compounds on the mean clinical EAE score of mice, in an experimental study according to the protocol shown in FIG. 5.

FIG. 7 comprises the results of an experimental study according to the protocol shown in FIG. 5, in the form of bar charts showing the effect of several steroid compounds on mice, in terms of the incidence of EAE disease (FIG. 7a), mean maximum clinical score (FIG. 7b) and day of onset of disease (FIG. 7c).

FIG. 8 comprises stained sections showing the effect of several steroid compounds on inflammation in the spinal cord of mice, in an experimental study according to the protocol shown in FIG. 5.

FIG. 9 comprises the results of an experimental study in the form of bar charts showing the effect of several steroid compounds on the lymph node cells of certain mice that had been induced to develop EAE, in terms of the cell proliferation to MOG₃₅₋₅₅ peptide (FIG. 9a), the amounts produced of IFN-γ and IL-10 (FIGS. 9b and 9c), the secretion of cytokine IL-17 (FIG. 9d) and the IFN-γ/IL-17 ratio (FIG. 9e).

FIG. 10 shows schematically a timeline for an experimental protocol for investigating the protective effect of several steroid compounds after the onset of experimental autoimmune encephalomyelitis (EAE).

FIG. 11 is a graph showing the effect over time of several steroid compounds on the mean clinical EAE score of mice, in an experimental study according to the protocol shown in FIG. 10.

FIG. 12 is a bar chart showing the effect of several steroid compounds on mice, in terms of the mean maximum clinical score, in an experimental study according to the protocol shown in FIG. 10.

FIG. 13 comprises the results of an experimental study in the form of bar charts showing the effect of several steroid compounds on the lymph node cells of certain mice that had been induced to develop EAE, in terms of the amounts produced of IL-10 (FIG. 13a), and the secretion of IFN-γ (FIG. 13b) and cytokine IL-17 (FIG. 13c).

FIG. 14 comprises the results of an experimental study of certain mice, in the form of bar charts showing the effect of several steroid compounds on the numbers of different types of leukocytes in the bronchoalveolar lavage (FIG. 14a), the lung leukocytic infiltration (FIG. 14b) and the OVA-specific T cell proliferation (FIG. 14c).

FIG. 15 shows schematically a timeline for an experimental protocol for investigating the ability of several steroid compounds to suppress established MOG peptide-induced experimental autoimmune encephalomyelitis (EAE).

FIG. 16 is a graph showing the effect over time of several steroid compounds on the mean clinical EAE score of mice, in an experimental study according to the protocol shown in FIG. 15.

FIG. 17 is a bar chart showing the effect of several steroid compounds on mice, in terms of the mean maximum clinical score, in an experimental study according to the protocol shown in FIG. 15.

FIG. 18 comprises stained sections showing the effect of certain steroid compounds on inflammation in the spinal cord of mice, in an experimental study according to the protocol shown in FIG. 15.

FIG. 19 comprises the results of an experimental study in the form of bar charts showing the effect of several steroid compounds on the lymph node cells of certain mice that had been induced to develop EAE, in terms of the cell proliferation to MOG₃₅₋₅₅ peptide (FIG. 19a), the amounts produced of IFN-γ and IL-17 (FIGS. 19b and 19c), and the secretion of cytokine IL-10 (FIG. 19d).

FIG. 20 is a bar chart showing the effect of several steroid compounds on the numbers of CD3⁺CD4⁺ IL10⁺ T cells in cultures after flow-cytometric analysis, in an experimental study.

FIG. 21 is a graph showing the effect over time of certain steroid compounds on the mean clinical EAE score of mice, in an experimental study.

FIG. 22 is a bar chart showing the effect of certain steroid compounds on mice, in terms of the mean maximum clinical score, in an experimental study.

FIG. 23 comprises the results of an experimental study in the form of bar charts showing the effect of certain steroid compounds on the lymph node cells of certain mice that had been induced to develop EAE, in terms of the cell proliferation to MOG₃₅₋₅₅ peptide (FIG. 23a) and the amounts of IFN-γ and IL-17 produced (FIGS. 23b and 23c).

FIG. 24 is a graph showing the effect over time of certain steroid compounds on the mean clinical EAE score of mice, in an experimental study.

FIG. 25 is a bar chart showing the effect of certain steroid compounds on mice, in terms of the mean maximum clinical score, in an experimental study.

FIG. 26 comprises the results of an experimental study in the form of bar charts showing the effect of certain steroid compounds on the lymph node cells of certain mice that had been induced to develop EAE, in terms of the cell proliferation to MOG₃₅₋₅₅ peptide (FIG. 26a) and the amounts of IFN-γ and IL-17 produced (FIGS. 26b and 26c).

FIG. 27 comprises the results of an experimental study in the form of bar charts showing the effect of certain steroid compounds on the numbers of CD11c⁺ cells (FIG. 27a), CD3⁺CD4⁺ IL17⁺ T cells (FIG. 27b) and CD4⁺ Foxp3⁺ T cells in cultures after flow-cytometric analysis, in an experimental study.

FIG. 28 is a graph showing the effect over time of certain steroid compounds on the mean clinical EAE score of mice, in an experimental study.

FIG. 29 is a bar chart showing the effect of certain steroid compounds on mice, in terms of the mean maximum clinical score, in an experimental study.

FIG. 30 is a bar chart showing the effect of certain steroid compounds on the lymph node cells of certain mice that had been induced to develop EAE, in terms of the cell proliferation to MOG₃₅₋₅₅ peptide.

FIG. 31 is a graph showing the effect over time of certain steroid compounds on the mean clinical EAE score of mice, in an experimental study.

FIG. 32 is a bar chart showing the effect of certain steroid compounds on mice, in terms of the mean maximum clinical score, in an experimental study.

FIG. 33 comprises the results of an experimental study in the form of bar charts showing the effect of certain steroid compounds on the lymph node cells of certain mice that had been induced to develop EAE, in terms of the cell proliferation to MOG₃₅₋₅₅ peptide (FIG. 33a) and the amounts of IFN-γ and IL-17 produced (FIGS. 33b and 33c).

FIG. 34 is a graph showing the effect over time of certain steroid compounds on the mean clinical EAE score of mice, in an experimental study.

FIG. 35 is a bar chart showing the effect of certain steroid compounds on mice, in terms of the mean maximum clinical score, in an experimental study.

FIG. 36 is a bar chart showing the effect of several steroid compounds on the numbers of CD4⁺ IL-17⁺ cells in cultures after flow-cytometric analysis, in an experimental study.

FIG. 37 is a bar chart showing the effect of several steroid compounds on the numbers of CD4⁺ IL-10⁺ cells in cultures after flow-cytometric analysis, in an experimental study.

FIG. 38 comprises output diagrams acquired by flow cytometry and showing the effect of several steroid compounds on the numbers of CD25 and Foxp3⁺ cells in cultures, in an experimental study.

FIG. 39 is a bar chart showing the effect of certain steroid compounds on naive CD4⁺ cells that had been cultured in an experimental study.

FIG. 40 is a bar chart showing the effect of certain steroid compounds on the lymph node cells of certain mice that had been first induced to develop EAE, treated with the steroid compounds and then immunized with OVA/CFA (Complete Freund's Adjuvant).

More detailed discussion of the drawings appears in Examples 1-8 below.

### DETAILED DESCRIPTION OF THE INVENTION

The present invention relates to the use of compounds of Formula I

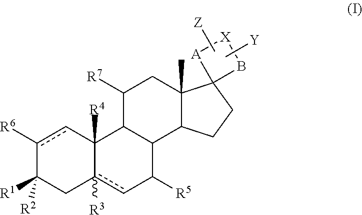

wherein

R¹ is hydroxyl, alkoxy, alkanoyloxy, aminocarbonyloxy or alkoxycarbonyloxy;

R² is hydrogen, optionally substituted alkyl, optionally substituted alkenyl, optionally substituted alkoxyalkyl, optionally substituted aminoalkyl, cyano, optionally substituted cyanoalkyl, optionally substituted thiocyanoalkyl, isothiocyano, optionally substituted azidoalkyl, optionally substituted alkanoyloxyalkyl, optionally substituted arylalkyl, optionally substituted heteroarylalkyl, optionally substituted arylalkenyl, optionally substituted heteroarylalkenyl, optionally substituted aryl, optionally substituted arylkynyl, optionally substituted arylkylalkynyl, optionally substituted alkanoyloxyalkynyl, optionally substituted heteroaryloxyalkynyl, optionally substituted oxoalkynyl or a ketal thereof, optionally substituted cyanoalkynyl, optionally substituted heteroarylalkynyl, optionally substituted hydroxyalkynyl, optionally substituted alkoxyalkynyl, optionally substituted aminoalkynyl, optionally substituted acylaminoalkynyl, optionally substituted mercaptoalkynyl, optionally substituted hydroxyalkynyl dioic acid hemi-ester or a salt thereof, or optionally substituted alkynyloxyalkynyl; or

R¹ is oxygen and R² is an alkyl or alkenyl or alkynyl group bonded to R¹ to form an oxygenated ring which can be optionally substituted;

R³ is hydrogen or, when a double bond is present between C5 and C6 of the steroid ring system, then R³ is not present;

R⁴ is hydrogen or lower alkyl;

R⁵ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;

R⁶ is hydrogen, amino, thio, sulfinyl, sulfonyl, sulfonamido, halogen, hydroxyl, optionally substituted alkoxy, optionally substituted alkyl, optionally substituted alkenyl, or optionally substituted alkynyl;

R⁷ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;

X is a valency bond, a methylene group (—CH₂—) or a heteroatom selected from oxygen, sulfur, or —NH, —S(O), —SO₂, —NR⁸, —NC(O)R⁸, —N-toluene-4-sulfonyloxy;

A is —(CH₂)ₙ—, a C₂₋₅ alkenylene group, or a C₂₋₅ alkynylene group, wherein n is an integer and can take the value of 0 or 1 or 2 or 3 or 4 or 5;

B is —(CH₂)_{y}—, a C₂₋₅ alkenylene group, or a C₂₋₅ alkynylene group, wherein y is an integer and can take the value of 1 or 2 or 3 or 4 or 5;

Y can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, or optionally substituted C(S)O—W;

Z can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, optionally substituted C(S)O—W;

Y and Z can be bonded to the same carbon of the spirocyclic substituent at C17;

W is optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloakenyl, optionally substituted C₂₋₁₀ alkynyl, optionally substituted heterocycloalkynyl, optionally substituted aryl, or optionally substituted heteroaryl;

R⁸ and R⁹ are independently optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocyclo-alkenyl, optionally substituted C₂₋₁₀ alkynyl, optionally substituted heterocyclo-alkynyl, optionally substituted aryl, or optionally substituted heteroaryl;

and the dotted lines indicate that a single or double bond may be present.

The compounds of Formula I and their pharmaceutically acceptable esters, salts or acid addition salts can be used for treating, preventing or ameliorating the symptoms of inflammatory conditions, in particular those associated with the immune system. Conditions that may be treated include, by way of example only, asthma, lung inflammation, retinal inflammatory conditions, autoimmune diseases such as rheumatoid arthritis, diabetes type I, systemic lupus erythematosus, myasthenia gravis, aplastic anaemia, autoimmune haemolytic anaemia, refractory scleroderma, dermatitis, acquired pemphigus, Grave's disease, autoimmune hepatitis, psoriasis, Crohn's disease, ulcerative colitis and inflammatory bowel diseases and myopathies. Multiple sclerosis is another condition that may be treated in accordance with the invention.

Preferred are embodiments of the invention wherein in Formula I above X is a methylene group, an oxygen atom or —NH. More preferably, X is an oxygen atom.

Also preferred are embodiments of the invention wherein in Formula I above a double bond is present between C5 and C6 of the steroid ring system, so that R³ is not present.

Also preferred are embodiments of the invention wherein in Formula I above R¹═OH; R²═R⁵═R⁶═R⁷═Y═H and R⁴=Me.

More preferred are embodiments of the invention wherein in Formula I above R¹═OH; R²═R⁵═R⁶═R⁷═Y═H, A=—(CH₂)ₙ— and B═—(CH₂)_{y}—; no double bond is present between C1 and C2 of the steroid ring system; a double bond is present between C5 and C6 of the steroid ring system, so that R³ is not present; and R⁴=Me. Yet more preferred are such compounds wherein n=0 and y=1.

Most preferred are embodiments of the invention wherein the compound of Formula I is selected from the following, including pharmaceutically acceptable esters, salts and acid addition salts thereof:
17β-spiro-[5-androsten-17,2′-oxiran]-3β-ol;
(20S)-3β,21-dihydroxy-17β,20-epoxy-5-pregnene;
(20S)-3β-hydroxy-17β,20-epoxy-20-(2-bromoethynyl)-5-androstene; and
3β,21-dihydroxy-17α,20-epoxy-5-pregnene.

The following terms, alone or in combination, are defined herein as follows:

The term “alkyl” herein denotes a straight chain or branched chain or cyclic saturated hydrocarbon group. Preferable are C₁-C₁₆ alkyl groups. Unless otherwise specifically limited, an alkyl group may be unsubstituted, singly substituted or, if possible, multiply substituted, with substituent groups in any possible position. Unless otherwise specifically limited, a cyclic alkyl group includes monocyclic, bicyclic, tricyclic and polycyclic rings, for example adamantyl, norbornyl and related terpenes.

The term “heterocycloalkyl” herein denotes a cyclic hydrocarbon group containing one, two, three or four O, N or S atoms or combinations of O, N, S atoms, e.g. oxiranyl, oxetanyl, tetrahydrofuranyl, tetrahydro-2H-pyranyl, morpholinyl, aziridinyl, azetidinyl, pyrrolidinyl, piperidinyl, tetrahydrothiophenyl, tetrahydro-2H-thiopyranyl. Unless otherwise specifically limited, a heterocycloalkyl group may be unsubstituted, singly substituted or, if possible, multiply substituted, with substituent groups in any possible position.

The term “haloalkyl” herein denotes an alkyl group substituted with one or more halogens.

The term “alkenyl”, alone or in combination, herein denotes a straight chain or branched chain or cyclic unsaturated hydrocarbon group which contains at least one carbon-carbon double bond. Unless otherwise specifically limited, an alkenyl group may be unsubstituted, singly substituted or, if possible, multiply substituted, with substituent groups in any possible position. Preferable are C₂-C₁₆ alkenyl groups. Alkenyl is meant to include the alkenyl group, which possesses two consecutive double bonds.

The term “heterocycloalkenyl” herein denotes a cyclic unsaturated hydrocarbon group containing at least one carbon-carbon double bond containing one, two, three or four O, N or S atoms or combinations of O, N, S atoms. Unless otherwise specifically limited, a heterocycloalkenyl group may be unsubstituted, singly substituted or, if possible, multiply substituted, with substituent groups in any possible position.

The term “alkynyl”, alone or in combination, herein denotes a straight chain or branched chain or cyclic unsaturated group which contains at least one carbon-carbon triple bond. Unless otherwise specifically limited, an alkynyl group may be unsubstituted, singly substituted or, if possible, multiply substituted, with substituent groups in any possible position. Preferable are C₂-C₁₆ alkynyl groups.

The term “aryl”, alone or in combination, herein denotes an aromatic group which contains at least one ring with conjugated π electrons, carbocyclic aryl groups, and biaryl groups which may be unsubstituted, singly substituted or, if possible, multiply substituted, with substituent groups in any possible position. Preferable are C₂-C₁₀ aryl groups. Typical aryl groups include phenyl, naphthyl, phenanthryl, anthracyl, indenyl, azulenyl, biphenyl, biphenylenyl and fluorenyl groups.

The term “biaryl” represents aryl groups substituted by other aryl groups.

The term “carbocyclic aryl” refers to groups wherein the ring atoms on the aromatic ring are carbon atoms.

The term “thio” herein denotes —SR¹⁰, where R¹⁰ is hydrogen, alkyl, alkenyl, alkynyl, aryl, arylalkyl or heteroaryl, all of which may be optionally substituted.

The term “sulfonyl” herein denotes —SOR¹⁰, where R¹⁰ is hydrogen, alkyl, alkenyl, alkynyl, aryl, arylalkyl or heteroaryl, all of which may be optionally substituted.

The term “sulfonyl” herein denotes —SO₂R¹⁰, where R¹⁰ is hydrogen, alkyl, alkenyl, alkynyl, aryl, arylalkyl or heteroaryl, all of which may be optionally substituted.

The term “sulfonamido” herein denotes —SO₂ NR¹⁰R¹¹, wherein R¹⁰ and R¹¹ are independently hydrogen, alkyl, alkenyl, alkynyl, aryl, arylalkyl or heteroaryl, all of which may be optionally substituted.

The term “optionally substituted” or “substituted” refers to groups substituted by a substituent group listed below, in any possible position. Substituent groups for the above moieties useful in the invention are those groups that do not significantly diminish the biological activity of the inventive compound. Substituent groups that do not significantly diminish the biological activity of the inventive compound include, for example, lower alkyl (acyclic and cyclic), aryl (carbocyclic aryl and heteroaryl), alkenyl, alkynyl, alkoxy, halo, haloalkyl, amino, alkylamino, dialkylamino, mercapto, alkylthio, alkylsulfinyl, alkylsulfonyl, nitro, alkanoyl, alkanoyloxy, alkanoyloxyalkanoyl, alkoxycarboxy, carbalkoxy, carboxamido, formyl, carboxy, hydroxy, cyano, azido, isocyano, isothiocyano, oxime, keto and cyclic ketals thereof, alkanoylamido, heteroaryloxy, O-aroyl, OalkylOH, OalkenylOH, OalkynylOH, OalkylNX₁X₂, OalkenylNX₁X₂, OalkynylNX₁X₂, NH-acyl, NH-aroyl, CF₃, COOX₃, SO₃H, PO₃X₁X₂, OPO₃X₁X₂, SO₂NX₁X₂, CONX₁X₂, wherein X₁ and X₂ each independently denotes H or alkyl or alkenyl or alkynyl, or X₁ and X₂ together comprise part of a heterocyclic ring having about 4 to about 7 ring atoms and optionally one additional heteroatom selected from O, N or S, or X₁ and X₂ together comprise part of an imide ring having about 5 to 6 ring atoms and X₃ denotes H, alkyl, alkenyl, alkynyl, hydroxy-lower alkyl or alkyl-NX₁X₂.

The term “lower” is referred to herein in connection with organic radicals or compounds containing one up to and including six carbon atoms. Such groups may be straight chain, branched chain, or cyclic.

The term “heteroaryl” refers to carbon-containing 5-14 membered cyclic unsaturated radicals containing one, two, three or four O, N or S atoms and having 6, or 14 π electrons delocalized in one or more rings, e.g., thienyl, benzo[b]thienyl, naphtha[2,3-b]thienyl, thianthrenyl, furyl, pyranyl, isobenzofuranyl, chromenyl, xanthenyl, phenoxanthinyl, 2H-pyrrolyl, imidazolyl, pyrazolyl, pyridyl, pyrimidinyl, pyridazinyl, indolizinyl, isoindolyl, 3H-indolyl, indoyl, indazolyl, purinyl, 4H-quinolizinyl, isoquinolyl, quinolyl, phthazinyl, naphthyridinyl, quinazolinyl, cinnolinyl, pterdinyl, 5aH-carbazoyl, carbozoyl, beta-carbolinyl, phenanthridinyl, acrindinyl, oxazolyl, pyrimidinyl, benzimidazolyl, triazolyl, each of which may be optionally substituted as discussed above.

The present invention also relates to pharmaceutically acceptable esters and salts of the compounds of Formula I, including acid addition salts.

Those skilled in the art will recognize that stereocentres exist in compounds of Formula I. Accordingly, the present invention includes all possible stereoisomers and geometric isomers of Formula I as a mixture or as pure diastereomers. When a compound of Formula I is desired as a single diastereomer, it may be obtained either by resolution of the final product or by stereospecific synthesis from either isomerically pure starting material or any convenient intermediate.

Included within the scope of the present invention are the crystalline forms (e.g. polymorphs), enantiomeric forms and tautomers of the compounds of Formula I as defined herein and of the pharmaceutically acceptable salts or acid addition salts thereof.

Some specific compounds of Formula I are:
1) 17β-spiro-[5-androsten-17,2′-oxiran]-3β-ol (“BNN-50”)
2) (20S)-3β,21-dihydroxy-17β,20-epoxy-5-pregnene (“BNN-124”)
3) (20S)-3β-hydroxy-17β,20-epoxy-20-(2-bromoethynyl)-5-androstene
4) 3β,21-dihydroxy-17α,20-epoxy-5-pregnene (“BNN-93”)

The compounds of Formula I may be prepared from commercially available steroid compounds using conventional synthetic reactions familiar to those skilled in the art. Preferred embodiments of the invention wherein X is an oxygen atom can be prepared from the important intermediate (20S)-3β-(t-butyldiphenylsilyloxy)-21-hydroxy-17β,20-epoxy-5-pregnene employing a series of synthetic steps in the appropriate order including but not limited to oxidation, Wittig reaction, reduction, hydrogenation, oxime formation, halogenation, carbon-carbon coupling reactions and removal of the protective group at C3. Suitable hydroxyl protective groups other than the t-butyldiphenylsilyloxy, can be employed. WO 2008/155534 describes a number of preparative methods, and the Examples thereof include detailed preparative methods for BNN-50, BNN-93, BNN-124, (20S)-3β-hydroxy-17β,20-epoxy-20-(2-bromoethynyl)-5-androstene, 3β,22-dihydroxy-17β,21-oxetanyl-5-pregnene and 17β-spiro-[3,6-hydroxy-5-androsten-17,2′-oxiran-7-ylideneaminooxy]-acetic acid.

In accordance with the present invention formulations may be administered in a standard manner for the treatment of the indicated conditions, including but not limited to oral, parenteral, sublingual, transdermal, rectal, or administration via inhalation or via buccal administration. Additionally, compositions may be formulated for parenteral administration by injection or continuous infusion. The compositions may be formulated as a slow release form or as a depot preparation. The route of administration may be any route that effectively transports the active compound to the desired site for it to exert its anti-inflammatory effects. Any person trained in the art may extend the former description to any other method of administration, not harming the recipient.

The term “treat” or “treatment” as used herein includes prophylaxis. The pharmaceutical compositions for use in this invention are prepared in conventional dosage unit forms by incorporating an active compound or a mixture of such compounds, with non-toxic pharmaceutical carrier according to accepted procedures in a non-toxic amount sufficient to produce the desired pharmacodynamic activity in a subject, animal or human. Preferably, the composition contains the active ingredient in an active, but non-toxic amount which depends on the specific biological activity desired and the condition of the patient.

The pharmaceutical carrier employed may be, for example, either a solid or a liquid. Representative solid carriers are lactose, terra alba, sucrose, talc, gelatin, agar, pectin, acacia, magnesium stearate, stearic acid, microcrystalline cellulose, polymer hydrogels and the like. Typical liquid carriers are propylene glycol, aqueous solutions of β-cyclodextrins, syrup, peanut oil and olive oil and the like emulsions. Similarly, the carrier or diluent may include any time-delay material well known in the art, such as glycerol monostearate or glycerol distearate alone or with wax, microcapsules, microspheres, liposomes, and/or hydrogels.

In the case of a solid carrier, the preparation can be plain milled, micronized or nanosized, in oil, tableted, placed in a hard gelatin or enteric-coated capsule in micronized powder or pellet form, or in the form of a troche, lozenge, or suppository. In the case of a liquid carrier, the preparation can be in the form of a liquid, such as an ampoule, or as an aqueous or nonaqueous liquid suspension mixed with pharmaceutically acceptable preservatives and the like. When low dosages are required, nasal spray, sublingual administration and timed released skin patches are also suitable pharmaceutical forms for topical administration.

### Experimental Section

The following Examples are provided for a better understanding of the invention, and are not to be taken as limiting the scope of the invention in any way.

In the following Examples it was investigated whether dehydroepiandrosterone (DHEA) and its related spiro compounds BNN-50, BNN-93 and BNN-124 (defined above) were suppressive against T cell responses ex vivo, as well as against immune-mediated diseases and responses in vivo, during experimental autoimmune encephalomyelitis and allergic airway inflammation.

### Example 1

### Activity of Synthetic Spirosteroids in Suppression of T Cell Responses Ex Vivo

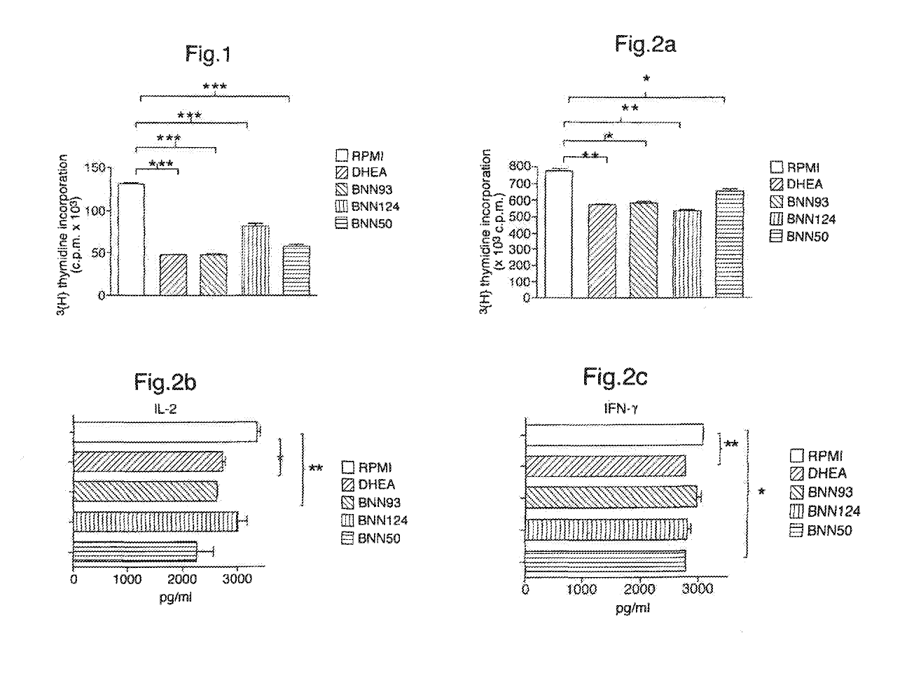

Lymph node (LN) cells were cultured from unprimed DO11.10 TCR Tg mice (which respond to OVA peptide 323-339) which were stimulated with OVA in the presence of DHEA or the spiro compounds BNN-50, BNN-93 and BNN-124 or the control RPMI. Treatment with DHEA and the spiro-analogs resulted in significantly decreased proliferation of the T cells that respond to the antigen (FIG. 1).

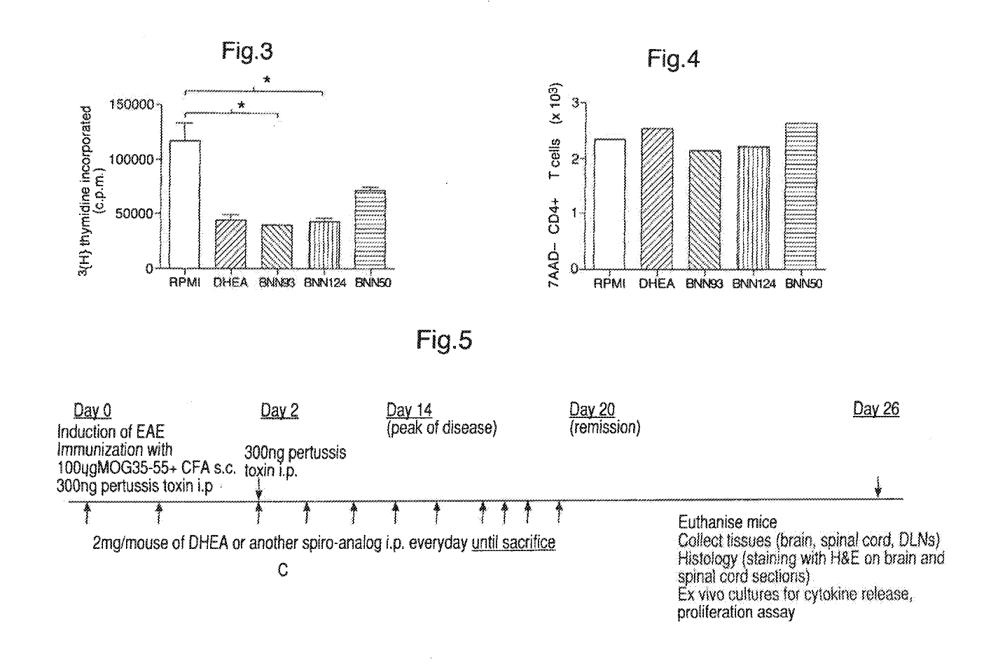

In order to test whether DHEA and the analogs exert their immunosuppressive effect directly on CD4⁺ T cells, purified CD4⁺ T cells were cultured from unprimed DO11.10 TCR Tg mice with mitomycin C-treated splenocytes and OVA peptide 323-339. Again, treatment with DHEA and the spiro compounds resulted in significantly decreased proliferation and secretion of IL-2 and IFN-γ (FIG. 2). The same suppressive effect of DHEA and the spiro compounds was observed when purified CD4⁺ T cells were stimulated from unprimed BALB/c mice with α-CD3/α-CD28 (non antigen-specific) (FIG. 3). The decreased proliferative response of CD4⁺ T cells was not due to increased cell death as evident by similar numbers of CD4⁺ 7AAD⁻ cells in all cultures after flow-cytometric analysis (FIG. 4).

Overall, the DHEA and the spiro compounds BNN-93, BNN-124, BNN-50 are seen to have an immunosuppressive effect on T cell responses in general.

### Example 2

### Ability of Synthetic Spirosteroids to Protect Against MOG Peptide-Induced Experimental Autoimmune Encephalomyelitis (EAE)

To evaluate the immunosuppressive effect of DHEA and the spiro compounds in vivo, an investigation was undertaken to establish whether DHEA and its spiro-analogs were protective against experimental autoimmune encephalomyelitis (EAE). EAE is an autoimmune condition of the central nervous system (CNS) and results from infiltration of the central nervous system by destructive autoreactive T lymphocytes and subsequent demyelination and axonal and neuronal degeneration.

Acute EAE was induced in C57BL/6 mice by immunizing them against the pathogenic myelin oligodendrocyte glycoprotein (MOG) peptide (amino acids 35-55). BNN-93, BNN-124, BNN-50 or DHEA (2 mg/mouse) or PBS (control) were administered intraperitoneally daily, from the day of EAE induction, until day 26, when mice were euthanized (FIG. 5). Mice were monitored daily in a blinded fashion for clinical symptoms (paralysis). Tissues (brain, spinal cord, DLNs) were collected in order to perform histological evaluation (staining with H&E on brain and spinal cord sections) and ex vivo cultures for measurement of T cell responses and cytokine release.

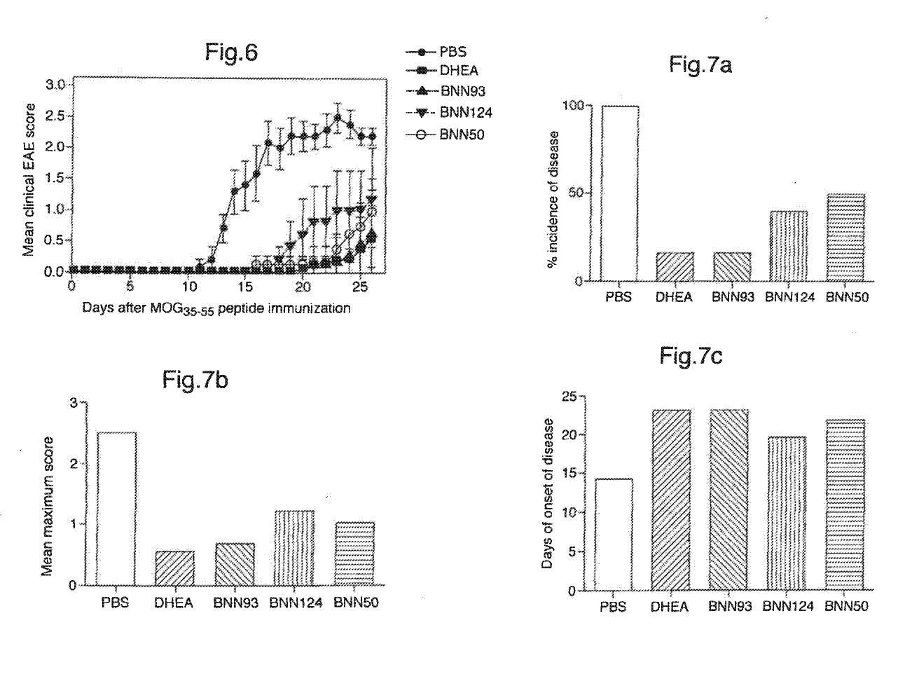

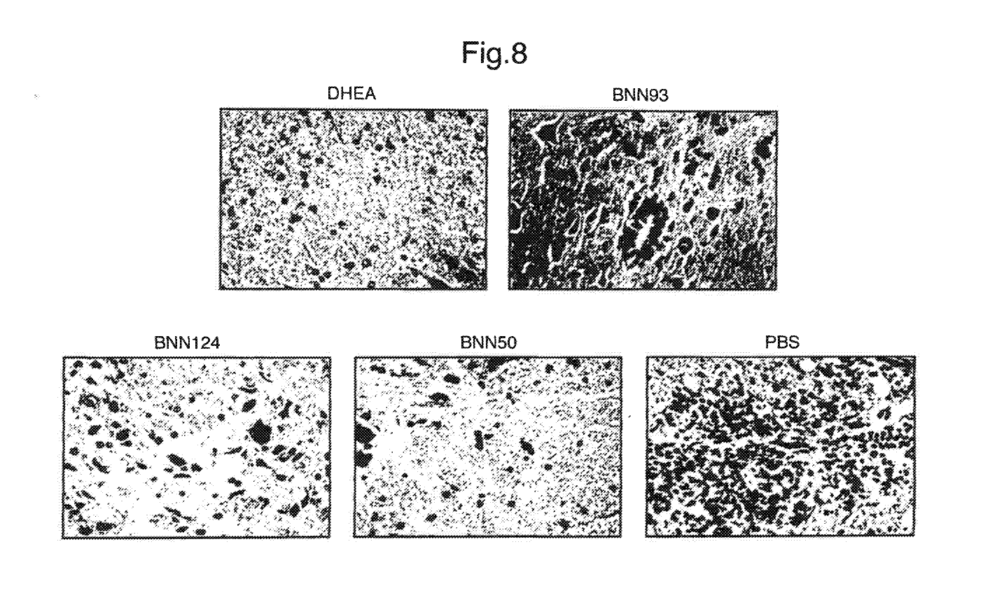

Administration of DHEA and the spiro compounds resulted in a decreased degree of paralysis. Overall, there was an improvement of the clinical course and a decrease in the mean maximum score (FIGS. 6 and 7). The decreased clinical score was associated with delayed onset and low incidence, in comparison to the control (FIG. 7). The protective effect of the DHEA and the spiro compounds was also associated with decreased inflammation in the spinal cord (FIG. 8).

Overall, the spiro compounds as well as DHEA conferred protection against MOG peptide-induced EAE and contributed to decreased clinical score and incidence, delayed disease onset and decreased inflammation in the CNS.

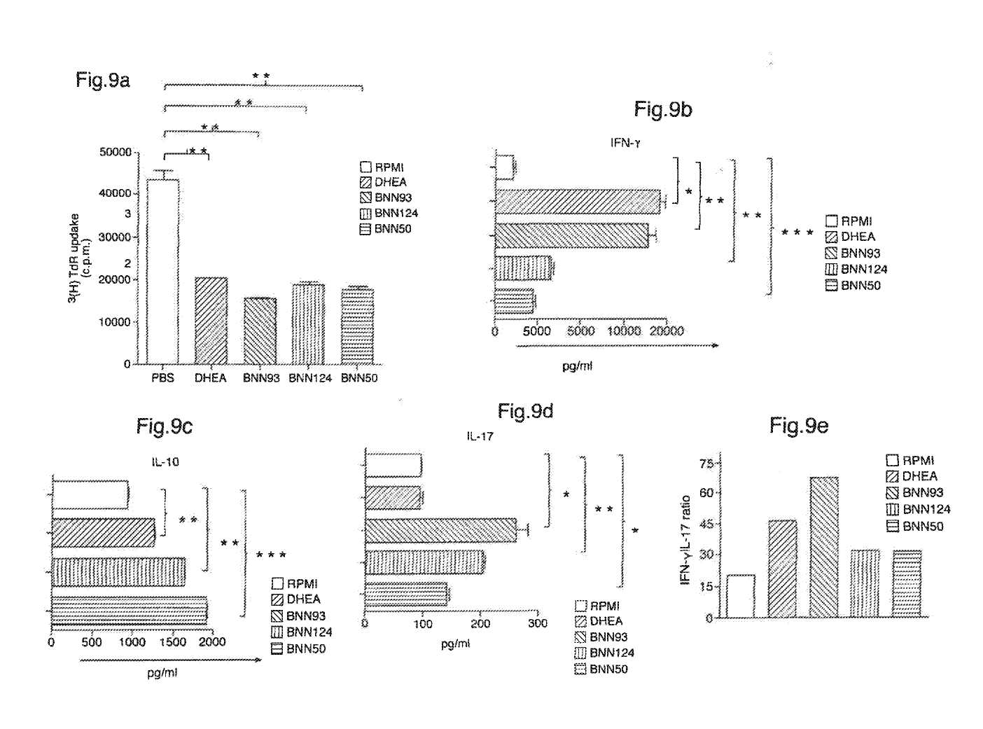

Also tested were the proliferative response and cytokine secretion of lymphoid cells (T cells) to MOG₃₅₋₅₅ peptide from the mice that had been induced to develop EAE. Draining lymph node cells from mice treated with DHEA and the spiro compounds showed significantly decreased proliferation to MOG₃₅₋₅₅ peptide (FIG. 9a) and produced substantially increased amounts of IFN-γ and IL-10 (FIGS. 9b and 9c), which are considered to have regulatory properties as indicated by several recent studies. In addition, the secretion of the inflammatory cytokine IL-17 was increased (FIG. 9d), but its absolute increase was extremely low in comparison to the IFN-γ increase. In fact, DHEA and the spiro compounds significantly increased the IFN-γ/IL-17 ratio (FIG. 9e), a finding consistent with a protective effect on EAE. Thus, DHEA and the spiro compounds may suppress EAE by shifting the balance of the highly pathogenic Th17 response towards a less pathogenic Th1 response.

Overall, these results show that treatment with DHEA and its spiro-analogs resulted in significantly suppressed pathogenic immune response.

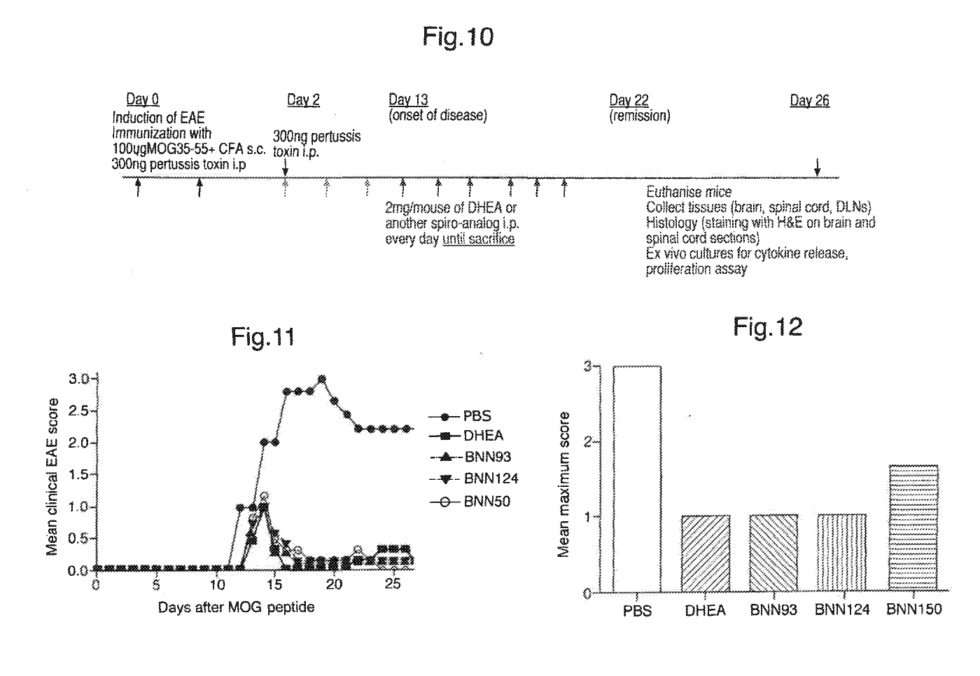

Tests were also carried out to discover whether DHEA and its spiro-analogs were protective after the onset of experimental autoimmune encephalomyelitis (EAE). For these tests, acute EAE was induced in C57BL/6 mice, as previously described. BNN-93, BNN-124, BNN-50 or DHEA (2 mg/mouse) or PBS were administered intraperitoneally daily, from the day of EAE onset, until day 26, when mice were euthanized (FIG. 10). Mice were monitored daily in a blinded fashion for clinical symptoms (paralysis). Tissues (brain, spinal cord, DLNs) were collected in order to perform histological evaluation (staining with H&E on brain and spinal cord sections) and ex vivo cultures for measurement of T cell responses and cytokine release.

Administration of DHEA and the spiro-compounds resulted in a decreased degree of paralysis. Overall, there was an improvement/reversal of the clinical course and a decrease in the mean maximum score (FIGS. 11 and 12).

Also tested were the cytokine secretion of lymphoid cells (T cells) to MOG₃₅₋₅₅ peptide from the mice that had been induced to develop EAE. Draining lymph node cells from mice treated with DHEA and the spiro compounds produced greatly increased amounts of IL-10 (FIG. 13a). In addition, the secretion of IFN-γ, as well as of the inflammatory cytokine IL-17 was substantially decreased (FIGS. 13b and 13c). Thus, DHEA and the spiro-compounds may protect after EAE onset by shifting the balance of the highly pathogenic Th17 response towards a T regulatory cell response characterized mainly by the production of IL-10.

### Example 3

### Activity of Synthetic Spirosteroids in Inhibiting TH-2 Immune Response and Protecting from Allergic Airway Inflammation

The in vivo effects of DHEA and the Spiro compounds were investigated during T_{H}2-mediated immune responses and subsequent disease development, using an established mouse model of allergic asthma. BALB/c mice were sensitized with 0.01 mg chicken ovalbumin (OVA) in 0.2 ml alum intraperitoneally on days 0 and 12, and were exposed to aerosolized OVA (5% for 20 min) on days 18-20. Mice were administered 2 mg/mouse of DHEA or the spiro compounds (BNN-93 or BNN-124 or BNN-50) or PBS on days 0, 1, 2, 12, 13, 14, 18, 19 and 20.

Mice treated with either DHEA or the spiro compounds had significantly decreased numbers of eosinophils and lymphomononuclear cells in the bronchoalveolar lavage (BAL), as compared to PBS-treated mice (FIG. 14a). Lung leukocytic infiltration was also significantly decreased in the mice treated with DHEA and the spiro compounds (FIG. 14b). Moreover, when the immune responses from the draining lymph nodes were examined, it was found that treatment with either DHEA or the spiro compounds resulted in significantly decreased OVA-specific T cell proliferation (FIG. 14c). In addition, levels of OVA-specific IgE, IgG1 and IgG2a were significantly decreased in mice treated with DHEA and the spiro compounds. Furthermore, mice treated with either DHEA or the spiro compounds had substantially increased numbers of CD3⁺CD4⁺ CD25⁺ Foxp3⁺ T cells (T regulatory cells) and suppressive CD11c⁺PDCA-1⁺ plasmacytoid dendritic cells (pDCs). Overall, these results provide evidence that DHEA and the spiro compounds suppress allergic airway inflammation, Th2 responses and B cell responses with concomitant induction of regulatory immune cell subsets.

### Example 4

### Activity of Synthetic Spirosteroids in Suppression of Established MOG Peptide-Induced EAE

An investigation was undertaken to establish whether DHEA and its spiro-analogs were effective in suppressing EAE that was already established in a mice population.

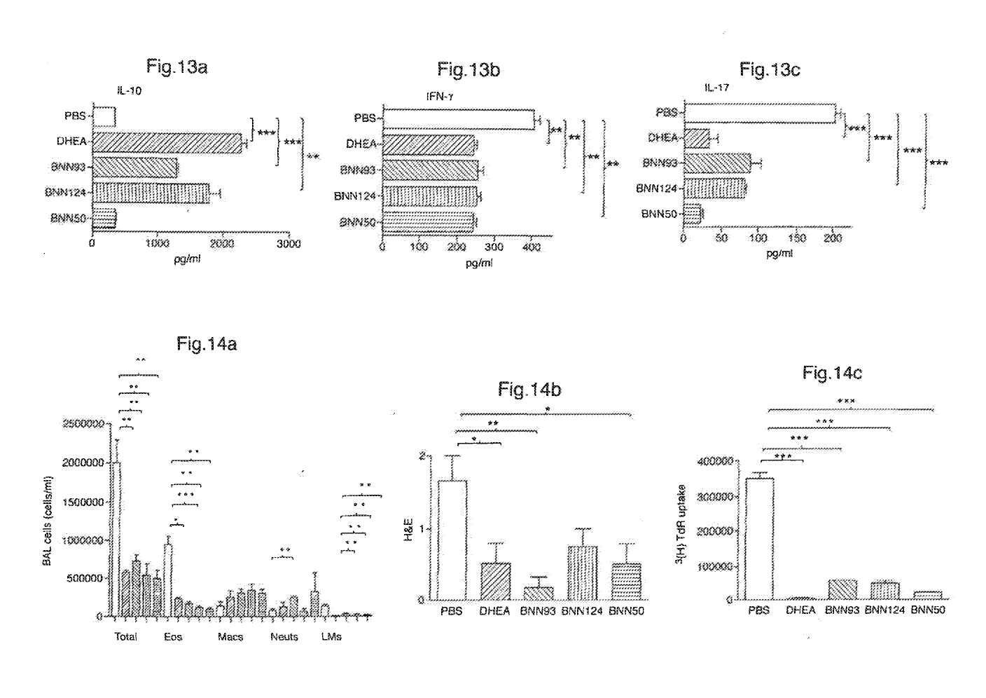

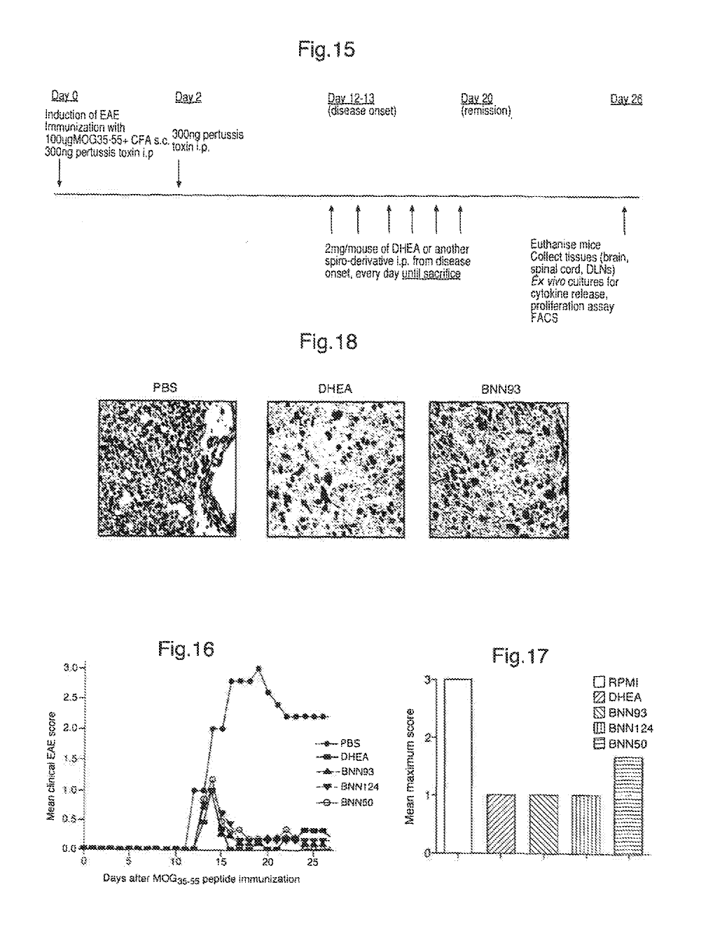

Acute EAE was induced in C57BL/6 mice by immunizing the animals against the pathogenic myelin oligodendrocyte glycoprotein (MOG) peptide (amino acids 35-55). Mice were left untreated until disease symptoms were apparent (score 1-2) at around day 12. BNN-93, BNN-124, BNN-50 or DHEA (2 mg/mouse) or PBS (control) were administered intraperitoneally daily, from day 13 after EAE induction, until day 26-28, when the mice were euthanized (FIG. 15). Mice were monitored daily in a blinded fashion for clinical symptoms (paralysis). Tissues (brain, spinal cord, DLNs) were collected in order to perform histological evaluation (staining with H&E on brain and spinal cord sections) and ex vivo cultures for measurement of T cell responses and cytokine release.

Administration of DHEA and the spiro compounds starting after disease onset resulted in a significantly decreased degree of paralysis. Overall, there was a significant improvement of the clinical course and a decrease in the mean maximum score (FIGS. 16 and 17). The protective effect of the DHEA and the spiro compounds was also associated with decreased inflammation in the spinal cord (FIG. 18).

Overall, the spiro compounds as well as DHEA suppressed ongoing MOG peptide-induced EAE and contributed to decreased clinical score and decreased inflammation in the CNS.

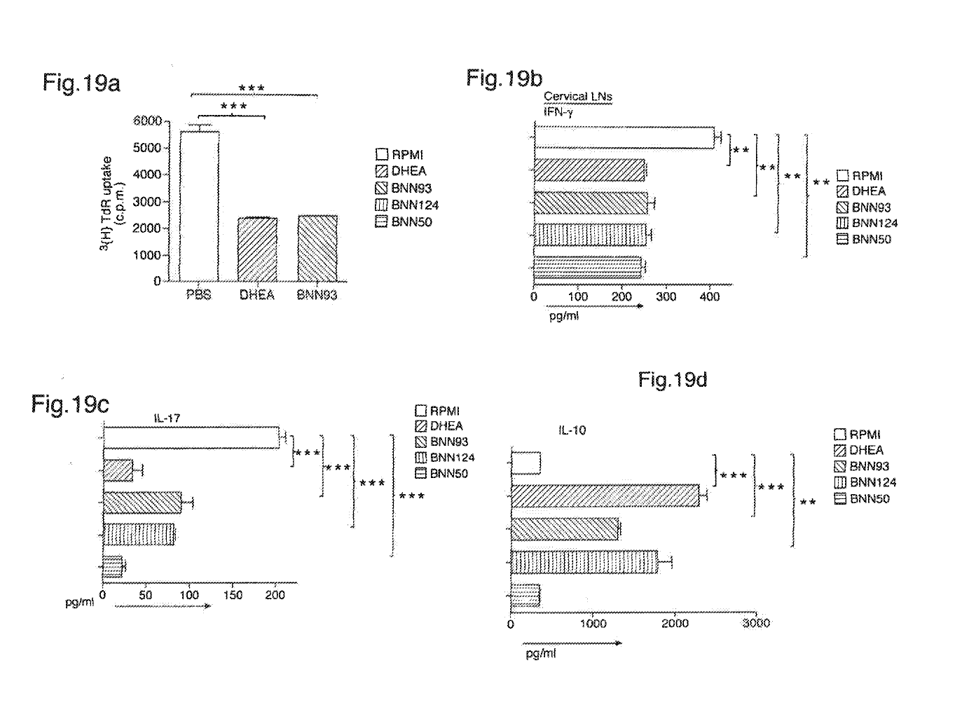

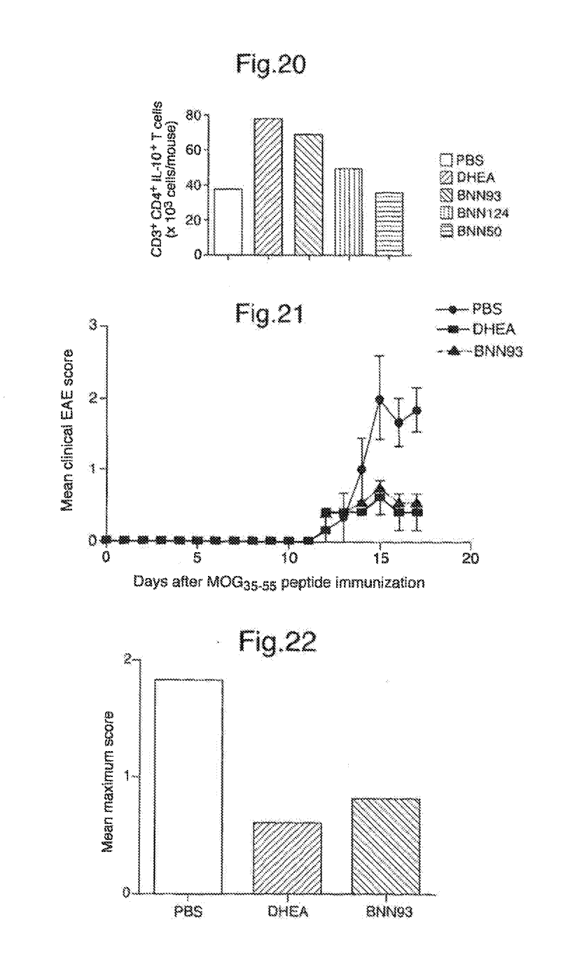

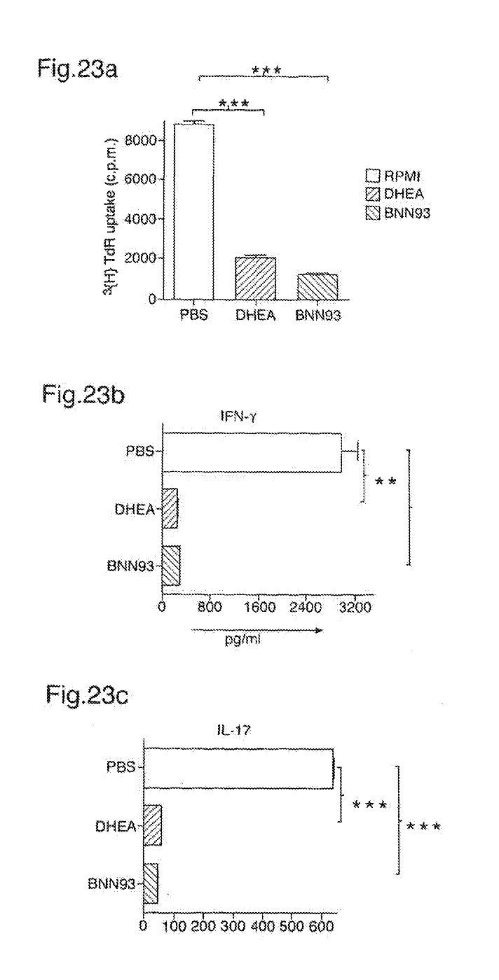

Also tested were the proliferative response and cytokine secretion of lymphoid cells (T cells) to MOG₃₅₋₅₅ peptide from the mice that had been induced to develop EAE. Draining lymph node cells from mice treated with DHEA and the spiro compounds showed significantly decreased proliferation to MOG₃₅₋₅₅ peptide (FIG. 19a) and produced substantially decreased amounts of IFN-γ and IL-17 (FIGS. 19b and 19c), which are considered to have inflammatory properties. This was accompanied by a significant reduction in the numbers of Th17 and Th1 cells in the draining lymph nodes and in the brain. In addition, the secretion of the immunoregulatory cytokine IL-10 was increased (FIG. 19d). Also, DHEA and the spiro compounds significantly increased the IL-10⁺ Treg cells, a finding consistent with a protective effect on EAE (FIG. 20). Thus, DHEA and the spiro compounds may suppress ongoing EAE by decreasing the pathogenic Th1 and Th17 immune response while increasing Treg cells. Similar results were obtained when mice were euthanized at disease peak (day 17) (FIGS. 21, 22 and 23a,b,c).

Overall, these results show that treatment of mice with ongoing EAE with DHEA and its spiro-analogs resulted in significantly suppressed pathogenic immune response and symptoms.

### Example 5

### Activity of Synthetic Spirosteroids in Suppression of Disease in Transferred Immune Cells

An experiment was undertaken to investigate whether immune cells in which EAE had been suppressed by DHEA and a spiro-analog would still have activity against EAE when adoptively transferred from one mice population to another.

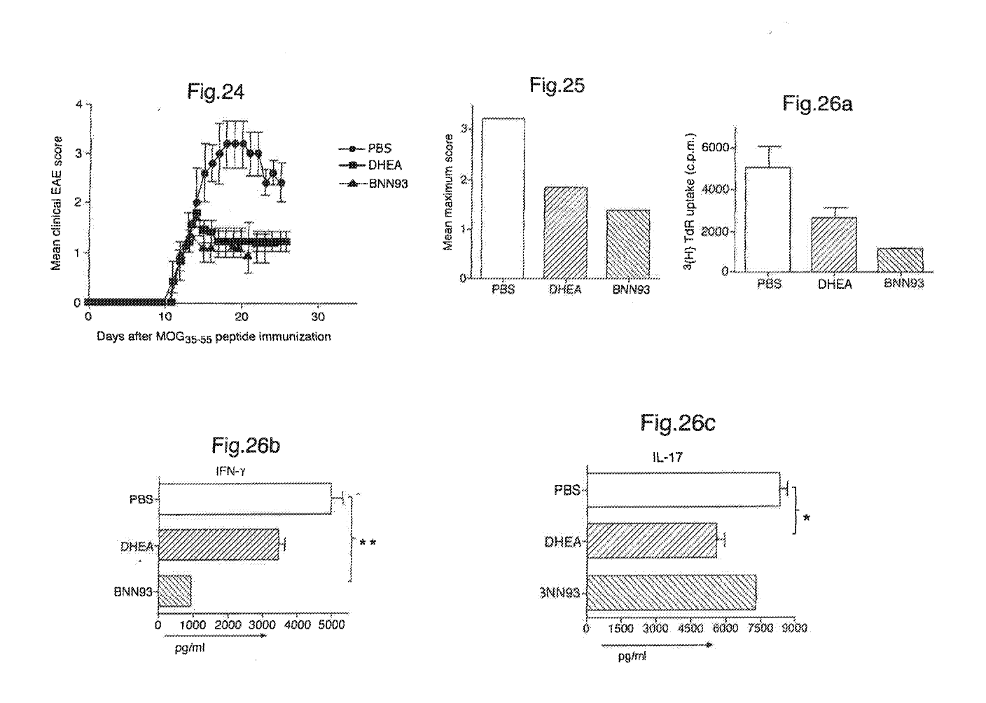

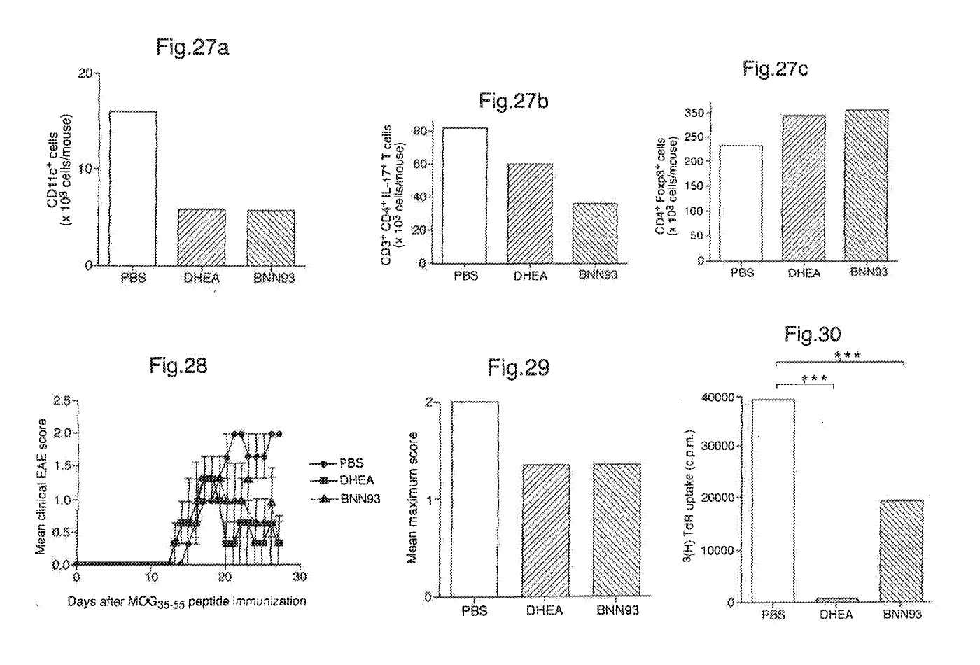

Draining lymph node (DLN) cells, obtained from mice with EAE which were treated with either DHEA or BNN-93 just after the disease onset (days 12-17), were adoptively transferred into recipients with ongoing EAE (around day 12). These DLN cells were found to confer suppression from EAE development, and resulted in a rapid recovery and a significant reduction in the mean maximum disease score, as compared to mice with EAE to which DLN cells from PBS-treated donors were adoptively transferred (FIGS. 24 and 25). The donor DLN cells were obtained on day 17, which is the peak of disease for control mice. This protection was observed until day 28-30 when the mice were euthanized. DLN cells obtained from mice adoptively transferred with DLN cells from DHEA- or BNN-93-treated donors with EAE exhibited significantly decreased ex vivo proliferation to MOG₃₅₋₅₅ and produced decreased levels of supernatant IFN-γ and IL-17 (FIGS. 26a,b,c). Additionally, the recruitment of CD11c⁺ dendritic cells that play a dominant role in the differentiation and activation of Th responses, as well as of the highly inflammatory IL-17 secreting T cells, was substantially decreased in the draining lymph nodes, while the recruitment of CD4⁺ Foxp3⁺ regulatory T cells was increased (FIGS. 27a,b,c).

The above results demonstrate that mice treated with DHEA and the spiro compounds develop immune cells that “carry” immunomodulatory capacity that can down-regulate ongoing disease upon transfer in recipients.

An investigation was also carried out to determine whether CD4+ T cells (T helper lymphocytes) from EAE-induced mice which were treated with DHEA, BNN-93 or PBS can transfer disease suppression. Adoptively transferred CD4⁺ T cells obtained from EAE-induced mice treated with DHEA or BNN-93 into recipients with ongoing EAE, conferred significant decrease of the mean maximum EAE score (FIGS. 28 and 29). In contrast, adoptive transfer of CD4⁺ T cells from PBS-treated EAE mice did not reduce disease. Accordingly, DLN cells and splenocytes obtained from mice adoptively transferred with DLN cells from DHEA- or BNN-93-treated EAE donors, exhibited significantly decreased proliferation to MOG₃₅₋₅₅ ex vivo (FIG. 30).

These results demonstrate that treatment with DHEA and the spiro compounds during EAE results in development of CD4+ T cells that can alone suppress ongoing EAE.

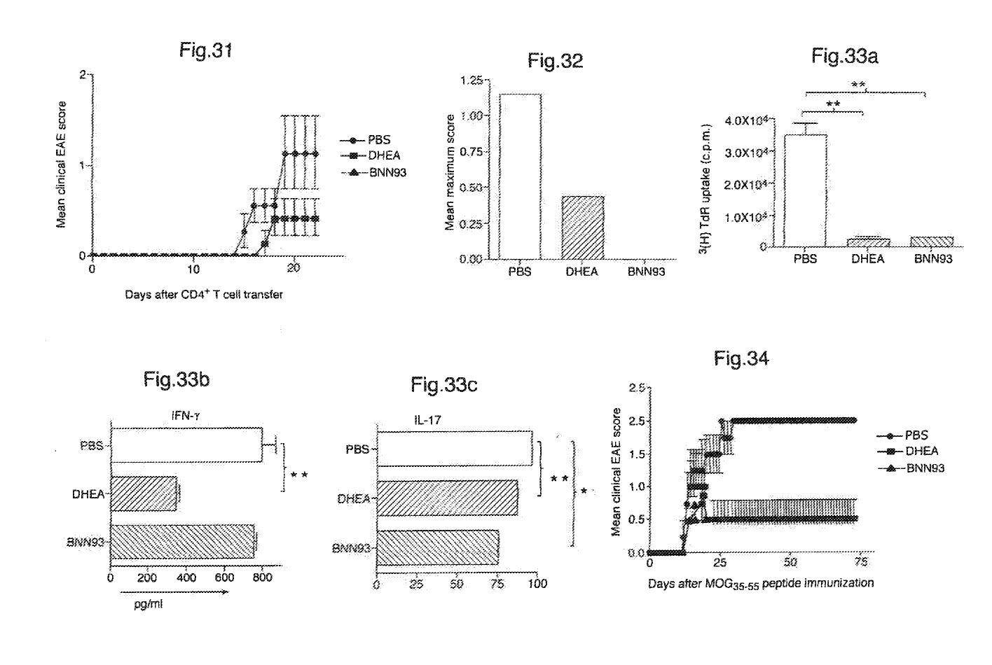

Adoptive transfer of CD4⁺ T cells from EAE-induced mice and treated with DHEA or BNN-93 into recipient mice that have no T and B cells (C57BL/6 Rag−/−) induced very low EAE scores to no disease (BNN-93) after pertussis toxin injections (FIGS. 31 and 32). In contrast, adoptive transfer of CD4⁺ T cells obtained from PBS-treated EAE-induced mice resulted in disease development in C57BL/6 Rag−/− recipients. DLN cells obtained from recipient mice adoptively transferred with CD4⁺ T cells from DHEA- or BNN-93-treated EAE donors, exhibited substantially decreased ex vivo proliferation to MOG₃₅₋₅₅ and decreased production of IFN-γ and IL-17 (FIGS. 33a,b,c). These results indicate that CD4+ T cells from treated mice are significantly less pathogenic.

### Example 6

### Activity of Synthetic Spirosteroids in Suppression of Disease Following Cessation of Treatment

An experiment was undertaken to investigate whether immune cells in which EAE had been suppressed by DHEA and a spiro-analog would continue in a mice population even after cessation of treatment.

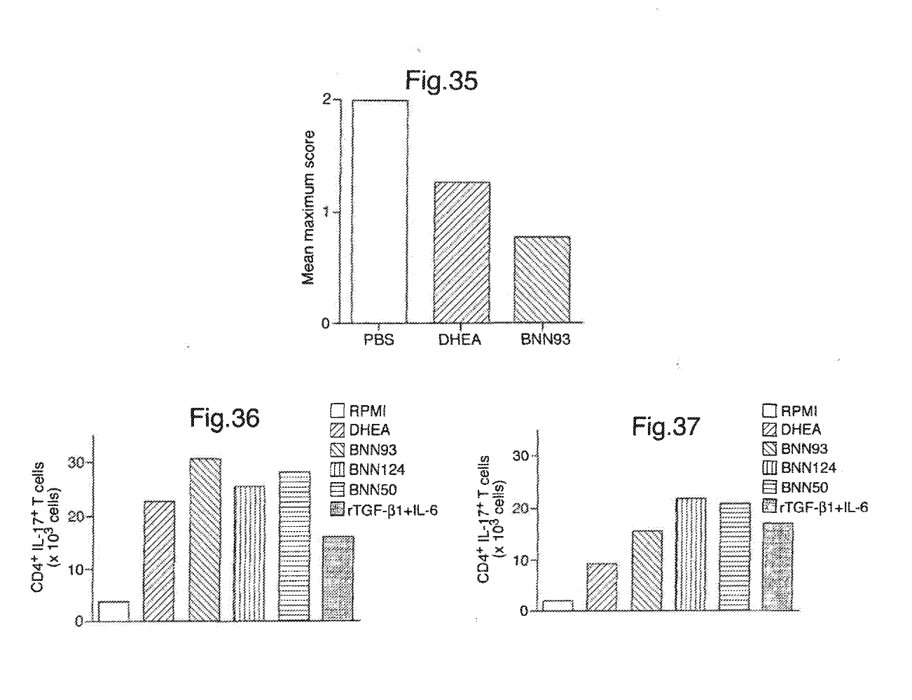

C57BL/6 mice were subjected to EAE induction in the same manner as described above, following which they were treated with DHEA, BNN-93 or PBS just after the disease onset (around day 13) for a period of 15 days, following which treatment was terminated. The mice continued to be monitored daily for clinical signs of EAE for 45 additional days thereafter. Mice that were previously treated with DHEA or BNN-93 for only 15 days remained protected from EAE development and their disease score remained unchanged, without any deterioration, as compared with control mice which continued to exhibit a high score disease course (FIGS. 34 and 35).

### Example 7

### Effect of Synthetic Spirosteroids on Th17 Cell Differentiation and Foxp3⁺ Treg Cell Generation

Naive CD4⁺ T cells were cultured in vitro under Th17 cell polarizing conditions (with rTGF-31 and rIL-6) and treated with DHEA, BNN-93, BNN-124, BNN-50 or RPMI (control culture medium) for 4 days. Treatment with DHEA and the spiro-analogs resulted in an increase in the number of CD4⁺ IL-17-secreting T cells (FIG. 36), and a significant increase in the number of CD4⁺ IL-10-secreting T cells, among Th17 differentiating cells, which are considered to have important immunoregulatory properties (FIG. 37).

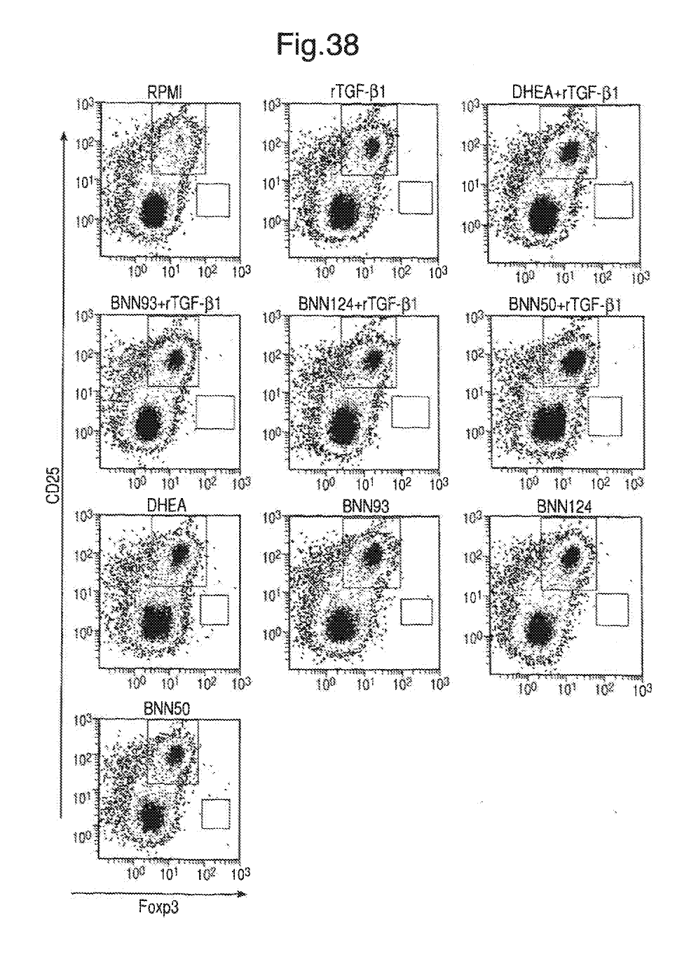

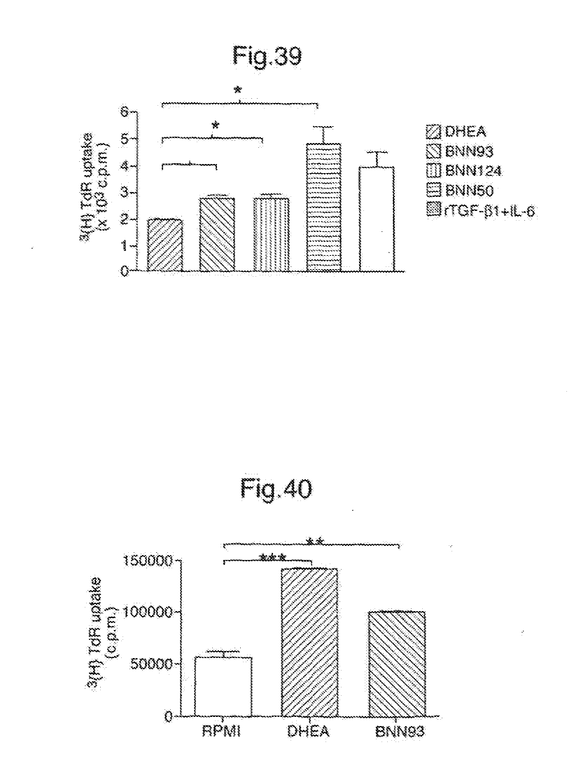

Additionally, naïve CD4⁺ T cells were cultured in vitro under Treg cell generating conditions (with rTGF-β1 and rIL-2) and treated with DHEA or BNN-93, BNN-124, BNN-50 or RPMI for 5 days. Treatment with DHEA and the spiro-analogs, with or even without rTGF-β1, resulted in a significant increase in the number of Foxp3⁺ Treg cells and increased proliferation (FIGS. 38 and 39).

These results demonstrate that DHEA and the spiro compounds influence the differentiation and generation of CD4⁺ T cell subsets with immunosuppressive capacity.

### Example 8

### Experiment Defining Responses to Another (Other than the MOG) Antigen

An experiment was undertaken to investigate the response of immune cells, in which EAE had been suppressed by DHEA and a spiro-analog, to different antigens involved in EAE development.

C57BL/6 mice were induced to develop EAE and were treated with DHEA, BNN93 or RPMI (control) for 15 days starting shortly after the disease onset. Treatment stopped and mice were then injected with OVA/CFA (Complete Freund's Adjuvant). Mice were monitored daily for clinical signs of EAE and those treated with DHEA or BNN93, which were clearly protected from EAE, had stable phenotype without any deterioration, in comparison to control mice.

After OVA/CFA immunization, mice treated with DHEA or BNN93 exhibited substantially increased T cell response to OVA ex vivo (shown as ³H-Thymidine incorporation) (FIG. 40), as well as increased production of IFN-γ. The results indicate that the neurosteroids are suppressive against EAE, but that they do not prevent the response of immune cells against an antigen other than those involved in EAE development. Instead, the response to a new antigen that the mice are exposed to is enhanced.

1. A method of preventing or treating an inflammatory condition, comprising administering to a patient an effective amount of a compound of Formula I 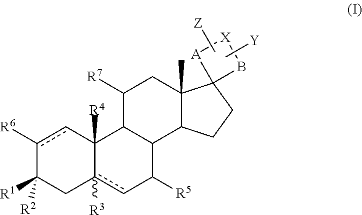
wherein
R¹ is hydroxyl, alkoxy, alkanoyloxy, aminocarbonyloxy or alkoxycarbonyloxy;
R² is hydrogen, optionally substituted alkyl, optionally substituted alkenyl, optionally substituted alkoxyalkyl, optionally substituted aminoalkyl, cyano, optionally substituted cyanoalkyl, optionally substituted thiocyanoalkyl, isothiocyano, optionally substituted azidoalkyl, optionally substituted alkanoyloxyalkyl, optionally substituted arylalkyl, optionally substituted heteroarylalkyl, optionally substituted arylalkenyl, optionally substituted heteroarylalkenyl, optionally substituted aryl, optionally substituted arylkynyl, optionally substituted arylkylalkynyl, optionally substituted alkanoyloxyalkynyl, optionally substituted heteroaryloxyalkynyl, optionally substituted oxoalkynyl or a ketal thereof, optionally substituted cyanoalkynyl, optionally substituted heteroarylalkynyl, optionally substituted hydroxyalkynyl, optionally substituted alkoxyalkynyl, optionally substituted aminoalkynyl, optionally substituted acylaminoalkynyl, optionally substituted mercaptoalkynyl, optionally substituted hydroxyalkynyl dioic acid hemi-ester or a salt thereof, or optionally substituted alkynyloxyalkynyl;
or
R¹ is oxygen and R² is alkyl or alkenyl or alkynyl group bonded to R¹ to form an oxygenated ring which can be optionally substituted;
R³ is hydrogen, or when a double bond is present between C5 and C6 of the steroid ring system, then R³ is not present;
R⁴ is hydrogen or lower alkyl;
R⁵ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
R⁶ is hydrogen, amino, thio, sulfinyl, sulfonyl, sulfonamido, halogen, hydroxyl, optionally substituted alkoxy, optionally substituted alkyl, optionally substituted alkenyl, or optionally substituted alkynyl;
R⁷ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
X is a valency bond, a methylene group (—CH₂—) or a heteroatom selected from oxygen, sulfur, or —NH, —S(O), —SO₂, —NR⁸, —NC(O)R⁸, —N-toluene-4-sulfonyloxy;
A is —(CH₂)ₙ—, a C₂₋₅ alkenylene group, or a C₂₋₅ alkynylene group, wherein n is an integer and can take the value of 0 or 1 or 2 or 3 or 4 or 5;
B is —(CH₂)_{y}—, a C₂₋₅ alkenylene group, or a C₂₋₅ alkynylene group, wherein y is an integer and can take the value of 1 or 2 or 3 or 4 or 5;
Y can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, or optionally substituted C(S)O—W;
Z can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, optionally substituted C(S)O—W;
Y and Z can be bonded to the same carbon of the spirocyclic substituent at C17;
W is optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
R⁸ and R⁹ are independently optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
and the dotted lines indicate that a single or double bond may be present;
or a pharmaceutically acceptable ester, salt or acid addition salt thereof.

2. A method according to claim 1 wherein X is an oxygen atom.

3. A method according to claim 1 wherein X is a methylene group.

4. A method according to claim 1 wherein X is —NH.

5. A method according to claim 1 wherein a double bond is present between C5 and C6 of the steroid ring system, so that R³ is not present.

6. A method according to claim 1 wherein R¹═OH; R²═R⁵═R⁶═R⁷═Y═H and R⁴=Me.

7. A method according to claim 1 wherein R¹═OH; R²═R⁵═R⁶═R⁷═Y═H, A=—(CH₂)ₙ—, B═—(CH₂)_{y}—; no double bond is present between C1 and C2 of the steroid ring system; a double bond is present between C5 and C6 of the steroid ring system, so that R³ is not present; R⁴=Me; n=0 and y=1.

8. A method according to claim 1 wherein the compound of Formula I is selected from the following, including pharmaceutically acceptable esters, salts and acid addition salts thereof:
17β-spiro-[5-androsten-17,2′-oxiran]-3β-ol;
(20S)-3β,21-dihydroxy-17β,20-epoxy-5-pregnene;
(20S)-3β-hydroxy-17β,20-epoxy-20-(2-bromoethynyl)-5-androstene;
3β,21-dihydroxy-17α,20-epoxy-5-pregnene.

9. A method as claimed in claim 1 wherein the condition is selected from the group consisting of asthma, lung inflammation, retinal inflammatory conditions.

10. A method as claimed in claim 1 wherein the condition is an autoimmune disease.

11. A method as claimed in claim 10 wherein the autoimmune disease is rheumatoid arthritis, diabetes type I, systemic lupus erythematosus, ulcerative colitis, inflammatory bowel disease or a myopathy.

12. A method as claimed in claim 10 wherein the autoimmune disease is myasthenia gravis, aplastic anaemia, autoimmune haemolytic anaemia, refractory scleroderma, dermatitis, acquired pemphigus, Grave's disease, autoimmune hepatitis, psoriasis or Crohn's disease.

13. A pharmaceutical composition to prevent or treat an inflammatory condition comprising an effective amount of a compound of Formula I and a pharmaceutical carrier, 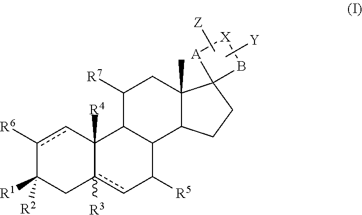
wherein
R¹ is hydroxyl, alkoxy, alkanoyloxy, aminocarbonyloxy or alkoxycarbonyloxy;
R² is hydrogen, optionally substituted alkyl, optionally substituted alkenyl, optionally substituted alkoxyalkyl, optionally substituted aminoalkyl, cyano, optionally substituted cyanoalkyl, optionally substituted thiocyanoalkyl, isothiocyano, optionally substituted azidoalkyl, optionally substituted alkanoyloxyalkyl, optionally substituted arylalkyl, optionally substituted heteroarylalkyl, optionally substituted arylalkenyl, optionally substituted heteroarylalkenyl, optionally substituted aryl, optionally substituted arylkynyl, optionally substituted arylkylalkynyl, optionally substituted alkanoyloxyalkynyl, optionally substituted heteroaryloxyalkynyl, optionally substituted oxoalkynyl or a ketal thereof, optionally substituted cyanoalkynyl, optionally substituted heteroarylalkynyl, optionally substituted hydroxyalkynyl, optionally substituted alkoxyalkynyl, optionally substituted aminoalkynyl, optionally substituted acylaminoalkynyl, optionally substituted mercaptoalkynyl, optionally substituted hydroxyalkynyl dioic acid hemi-ester or a salt thereof, or optionally substituted alkynyloxyalkynyl;
or
R¹ is oxygen and R² is alkyl or alkenyl or alkynyl group bonded to R¹ to form an oxygenated ring which can be optionally substituted;
R³ is hydrogen, or when a double bond is present between C5 and C6 of the steroid ring system, then R³ is not present;
R⁴ is hydrogen or lower alkyl;
R⁵ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
R⁶ is hydrogen, amino, thio, sulfinyl, sulfonyl, sulfonamido, halogen, hydroxyl, optionally substituted alkoxy, optionally substituted alkyl, optionally substituted alkenyl, or optionally substituted alkynyl;
R⁷ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
X is a valency bond, a methylene group —(CH₂)— or a heteroatom selected from oxygen, sulfur or, —NH, —S(O), —SO₂, —NR⁸, —NC(O)R⁸, —N-toluene-4-sulfonyloxy;
A is —(CH₂)ₙ—, a C₂₋₅ alkenylene group, or a C₂₋₅ alkynylene group, wherein n is an integer and can take the value of 0 or 1 or 2 or 3 or 4 or 5;
B is —(CH₂)_{y}—, a C₂₋₅ alkenylene group, or a C₂₋₅ alkynylene group, wherein y is an integer and can take the value of 1 or 2 or 3 or 4 or 5;
Y can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, or optionally substituted C(S)O—W;
Z can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, optionally substituted C(S)O—W;
Y and Z can be bonded to the same carbon of the spirocyclic substituent at C17;
W is optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
R⁸ and R⁹ are independently optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
and the dotted lines indicate that a single or double bond may be present;
or a pharmaceutically acceptable ester, salt or acid addition salt thereof.

14. A pharmaceutical composition of claim 13, wherein the inflammatory condition is asthma, lung inflammation, or a retinal inflammatory condition.

15. A pharmaceutical composition to prevent or treat an autoimmune disease comprising an effective amount of a compound of Formula I and a pharmaceutical carrier, 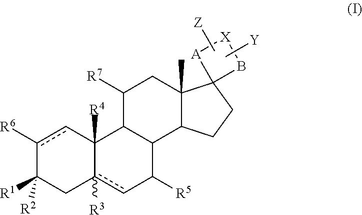
wherein
R¹ is hydroxyl, alkoxy, alkanoyloxy, aminocarbonyloxy or alkoxycarbonyloxy;
R² is hydrogen, optionally substituted alkyl, optionally substituted alkenyl, optionally substituted alkoxyalkyl, optionally substituted aminoalkyl, cyano, optionally substituted cyanoalkyl, optionally substituted thiocyanoalkyl, isothiocyano, optionally substituted azidoalkyl, optionally substituted alkanoyloxyalkyl, optionally substituted arylalkyl, optionally substituted heteroarylalkyl, optionally substituted arylalkenyl, optionally substituted heteroarylalkenyl, optionally substituted aryl, optionally substituted arylkynyl, optionally substituted arylkylalkynyl, optionally substituted alkanoyloxyalkynyl, optionally substituted heteroaryloxyalkynyl, optionally substituted oxoalkynyl or a ketal thereof, optionally substituted cyanoalkynyl, optionally substituted heteroarylalkynyl, optionally substituted hydroxyalkynyl, optionally substituted alkoxyalkynyl, optionally substituted aminoalkynyl, optionally substituted acylaminoalkynyl, optionally substituted mercaptoalkynyl, optionally substituted hydroxyalkynyl dioic acid hemi-ester or a salt thereof, or optionally substituted alkynyloxyalkynyl;
or
R¹ is oxygen and R² is alkyl or alkenyl or alkynyl group bonded to R¹ to form an oxygenated ring which can be optionally substituted;
R³ is hydrogen, or when a double bond is present between C5 and C6 of the steroid ring system, then R³ is not present;
R⁴ is hydrogen or lower alkyl;
R⁵ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
R⁶ is hydrogen, amino, thio, sulfinyl, sulfonyl, sulfonamido, halogen, hydroxyl, optionally substituted alkoxy, optionally substituted alkyl, optionally substituted alkenyl, or optionally substituted alkynyl;
R⁷ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
X is a valency bond a methylene group —CH₂— or a heteroatom selected from oxygen, sulfur, or —NH, —S(O), —SO₂, —NR⁸, —NC(O)R⁸, —N-toluene-4-sulfonyloxy;
A is —(CH₂)ₙ—, a C₂₋₅ alkenylene group, or a C₂₋₅ alkynylene group, wherein n is an integer and can take the value of 0 or 1 or 2 or 3 or 4 or 5;
B is —(CH₂)_{y}—, a C₂₋₅ alkenylene group, or a C₂₋₅ alkynylene group, wherein y is an integer and can take the value of 1 or 2 or 3 or 4 or 5;
Y can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, or optionally substituted C(S)O—W;
Z can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, optionally substituted C(S)O—W;
Y and Z can be bonded to the same carbon of the spirocyclic substituent at C17;
W is optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
R⁸ and R⁹ are independently optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
and the dotted lines indicate that a single or double bond may be present;
or a pharmaceutically acceptable ester, salt or acid addition salt thereof or a pharmaceutically acceptable ester, salt or acid addition salt thereof.

16. A pharmaceutical composition of claim 15, wherein the autoimmune disease is rheumatoid arthritis, diabetes type I, systemic lupus erythematosus, ulcerative colitis, inflammatory bowel disease or a myopathy.

17. A pharmaceutical composition of claim 15, wherein the autoimmune disease is myasthenia gravis, aplastic anaemia, autoimmune haemolytic anaemia, refractory scleroderma, dermatitis, acquired pemphigus, Grave's disease, autoimmune hepatitis, psoriasis or Crohn's disease.

18.-23. (canceled)

24. A method of claim 25 wherein the immunological response is T-cell activity in a human or non-human animal body.

25. A method of controlling or suppressing an immunological response of a human or non-human animal body, comprising administering to a patient an effective amount of a compound of Formula I 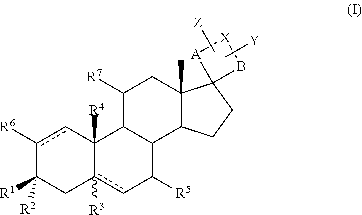
wherein
R¹ is hydroxyl, alkoxy, alkanoyloxy, aminocarbonyloxy or alkoxycarbonyloxy;
R² is hydrogen, optionally substituted alkyl, optionally substituted alkenyl, optionally substituted alkoxyalkyl, optionally substituted aminoalkyl, cyano, optionally substituted cyanoalkyl, optionally substituted thiocyanoalkyl, isothiocyano, optionally substituted azidoalkyl, optionally substituted alkanoyloxyalkyl, optionally substituted arylalkyl, optionally substituted heteroarylalkyl, optionally substituted arylalkenyl, optionally substituted heteroarylalkenyl, optionally substituted aryl, optionally substituted arylkynyl, optionally substituted arylkylalkynyl, optionally substituted alkanoyloxyalkynyl, optionally substituted heteroaryloxyalkynyl, optionally substituted oxoalkynyl or a ketal thereof, optionally substituted cyanoalkynyl, optionally substituted heteroarylalkynyl, optionally substituted hydroxyalkynyl, optionally substituted alkoxyalkynyl, optionally substituted aminoalkynyl, optionally substituted acylaminoalkynyl, optionally substituted mercaptoalkynyl, optionally substituted hydroxyalkynyl dioic acid hemi-ester or a salt thereof, or optionally substituted alkynyloxyalkynyl;
or
R¹ is oxygen and R² is alkyl or alkenyl or alkynyl group bonded to R¹ to form an oxygenated ring which can be optionally substituted;
R³ is hydrogen, or when a double bond is present between C5 and C6 of the steroid ring system, then R³ is not present;
R⁴ is hydrogen or lower alkyl;
R⁵ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
R⁶ is hydrogen, amino, thio, sulfinyl, sulfonyl, sulfonamido, halogen, hydroxyl, optionally substituted alkoxy, optionally substituted alkyl, optionally substituted alkenyl, or optionally substituted alkynyl;
R⁷ is hydrogen, amino, optionally substituted alkylamino, optionally substituted dialkylamino, optionally substituted alkenyl amino, optionally substituted dialkenyl-amino, optionally substituted alkynylamino, optionally substituted dialkynylamino, amido, thio, sulfinyl, sulfonyl, sulfonamido, halogen, hydroxyl, optionally substituted alkoxy, optionally substituted alkenyloxy, optionally substituted alkynyloxy alkyl, optionally substituted alkenyl, optionally substituted alkynyl, optionally substituted aryl, azido, optionally substituted heteroaryl, oxime ═N—O—R⁸, carboxymethyloxime, carboxyethyloxime, or carboxypropyloxime;
X is a valency bond, a methylene group (—CH₂—) or a heteroatom selected from oxygen, sulfur, or —NH, —S(O), —SO₂, —NR^{B}, —NC(O)R⁸, —N-toluene-4-sulfonyloxy;
A is —(CH₂)ₙ—, a C₂₋₅ alkenylene group, or a C₂₋₅ alkynylene group, wherein n is an integer and can take the value of 0 or 1 or 2 or 3 or 4 or 5;
B is —(CH₂)_{y}—, a C₂₋₅ alkenylene group, or a C₂₋₅ alkynylene group, wherein y is an integer and can take the value of 1 or 2 or 3 or 4 or 5;
Y can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, or optionally substituted C(S)O—W;
Z can be bonded to any carbon of the spirocyclic substituent at C17 of the steroid skeleton and is independently H, optionally substituted C₁₋₁₀ alkyl, an optionally substituted fused bicyclic ring system, an optionally substituted bridged bicyclic ring system, an optionally substituted bridged tricyclic ring system, optionally substituted C₂₋₁₀ alkenyl, optionally substituted C₂₋₁₀ alkynyl, optionally substituted aryl, optionally substituted arylalkyl, optionally substituted heteroaryl, optionally substituted heteroarylalkyl, formyl, carboxy, —NC(O)R⁸, NC(S)R⁸, —NR⁸R⁹, optionally substituted C(O)—W, optionally substituted C(O)O—W, optionally substituted C(S)O—W;
Y and Z can be bonded to the same carbon of the spirocyclic substituent at C17;
W is optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
R⁸ and R⁹ are independently optionally substituted C₁₋₁₀ alkyl, optionally substituted heterocycloalkyl, an optionally substituted fused bicyclic ring system, an optionally substituted bridged bicyclic ring system, an optionally substituted bridged tricyclic ring system, optionally substituted C₂₋₁₀ alkenyl, optionally substituted heterocycloalkenyl, optionally substituted C₂₋₁₀ alkynyl, optionally substituted heterocycloalkynyl, optionally substituted aryl, or optionally substituted heteroaryl;
and the dotted lines indicate that a single or double bond may be present;
or a pharmaceutically acceptable ester, salt or acid addition salt thereof.
